(19) United States
(12) Patent Application Publication
(10) Pub. No.: US 2019/0103175 A1
(43) Pub. Date: Apr. 4, 2019

(54) MEASUREMENT SYSTEM AND METHOD FOR DEFINING AND DETERMINING AN OVARIAN RESERVE

(71) Applicant: Celmatix Inc., New York, NY (US)

(72) Inventors: Piraye Yurttas Beim, New York, NY (US); Karen Hunter Cohn, New York, NY (US); Anthony Santistevan, New York, NY (US)

(21) Appl. No.: 15/963,563

(22) Filed: Apr. 26, 2018

Related U.S. Application Data
(60) Provisional application No. 62/490,513, filed on Apr. 26, 2017.

(51) Int. Cl. G16H 10/60; A61K 38/24; G16H 50/30; G16H 50/50; G16H 50/70

(52) U.S. Cl. CPC G16H 10/60; A61K 38/24; G16H 50/30; A61B 17/425; G16H 50/50; G16H 50/70; G06F 19/12

(57) ABSTRACT

Embodiments of the present disclosure relate an ovarian characteristic measurement method and system. A data interface is configured to receive a set of reproductive health data of a patient. The reproductive health data is processed using an ovarian characteristic data structure stored in memory. The data structure is generated from an ovarian characteristic data model that defines the ovarian characteristic. For instance, the data model defines a processing of the reproductive health data to yield a measurement of the ovarian characteristic. Accordingly, a format of the ovarian data structure is selected to optimize computing resources required to process the set of reproductive health data. The method and system then determine a value of the ovarian characteristic of the patient based on the processing of the set of reproductive health data.

### CROSS-REFERENCE TO RELATED APPLICATIONS

This application claims the benefit of and priority to U.S. Provisional Application Ser. No. 62/490,513, filed Apr. 26, 2017, the entire contents of which are incorporated by reference herein.

### BACKGROUND

The ovary is generally thought of as an egg bank from which a woman draws during her reproductive life. Ovarian reserve is a term that is used to determine the capacity of the ovary to provide egg cells that are capable of fertilization resulting in a healthy and successful pregnancy. The size of the initial ovarian reserve is strongly influenced by genetics.

Unfortunately, regardless of a woman's genetics, the ovarian reserve can be negatively affected by certain biological factors. For example, elevated androgen levels during prenatal development adversely affect women's initial ovarian reserve. Additionally, the number of egg cell that can be successfully recruited for a possible pregnancy declines with advanced maternal age. This decline constitutes a major factor in the inverse correlation between age and female fertility. One additional contributory mechanism for the decline in the ovarian reserve with age appears to be a decreased gene expression of proteins involved in DNA repair by homologous recombination such as BRCA1, MRE11, Rad51 and ATM.

Ovarian reserve testing is commonly administered to women who have been unsuccessful with their attempts to achieve pregnancy. Clinicians then use the results of the ovarian testing to provide individual women with a reproductive health diagnosis, corresponding treatment plan, and prognosis. While there is no known method for directly assessing the ovarian reserve of a woman, tests are available to indirectly estimate a woman's ovarian reserve. Example indirect tests include tests that measure Anti-Müllerian hormone (AMH), basal antral follicle count (BAFC), and day three levels of follicle-stimulating hormone (FSH), luteinizing hormone (LH), and estrogen (i.e., estradiol (E2)).

AMH is a substance produced by granulosa cells in ovarian follicles. An ovarian follicle is a roughly spheroid cellular aggregation set found in the ovaries. It secretes hormones that influence stages of the menstrual cycle. Women begin puberty with about 400,000 follicles each with the potential to release an egg cell (ovum) at ovulation for fertilization. Accordingly, a pool of ovarian follicles is believed to be closely related to ovarian reserve. As such, AMH blood levels are though to reflect the size of the remaining egg supply (i.e., ovarian reserve).

BAFC is a transvaginal ultrasound study that is used to measure a woman's ovarian reserve. An antral (resting) follicle is a small, fluid-filled sac that contains an immature egg. Each egg can potentially develop and ovulate. Because antral follicles are small follicles (about 2-9 mm in diameter) they can be seen, measured, and counted by using ultrasound imaging. As such, a number of antral follicles can be used to determine ovarian reserve.

FSH is a hormone produced by the pituitary gland. FSH causes follicles—the sacs in your ovaries that contain eggs—to grow. As such, FSH is the primary hormone responsible for producing eggs. The pituitary gland releases FSH to get a follicle “going” at the beginning of every menstrual cycle. If there are less follicles left (and perhaps lower quality follicles) the amount of FSH has to be increased to get a follicle developing. Therefore, high levels of FSH indicate a poor ovarian reserve as opposed to low levels of FSH which indicate a good ovarian reserve. However, low levels of FSH can indicate secondary ovarian failure (e.g., ovarian failure due to inappropriate regulatory signals (hypothalamic or pituitary pathology)).

LH is a hormone produced by gonadotropic cells in the anterior pituitary gland. In women, an acute rise of LH (“LH surge”) triggers ovulation and development of the corpus luteum. LH is known to work with FSH. FSH stimulates the ovarian follicle, causing an egg to grow. It also triggers the production of estrogen in the follicle. The rise in estrogen signals the pituitary gland to stop producing FSH and to start producing LH. This shift to LH causes an egg to be released from the ovary, in a process called ovulation. In the empty follicle, cells proliferate, turning it into a corpus luteum. This structure releases progesterone, a hormone necessary to maintain pregnancy. If pregnancy doesn't occur, the levels of progesterone drop off and the cycle begins again. High levels of LH can indicate that a woman is in menopause. For women younger than 40, high LH levels can suggest premature menopause. On the other hand, low LH levels can prevent ovulation and this pregnancy and indicates secondary ovarian failure.

An E2 test is a blood test that measures an amount of estradiol in a woman's blood. Estradiol is a form of the steroid hormone estrogen, and it's also called “17 beta-estradiol.” It is secreted into circulation by granulosa cells of developing ovarian follicles. For ovarian reserve testing, clinicians typically assess E2 levels early in the menstrual cycle, at days 2 or 3. A high E2 level suggests impaired oocyte development early in the menstrual cycle—a worrisome sign of reproductive aging and/or poor ovarian reserve. In addition, studies have reported that low levels of E2 reported lower success of fertility treatments.

Unfortunately, each of these tests have a degree of error associated with them because, as stated above, they are only indirect methods of determining ovarian reserve and, as such, cannot truly be validated. For instance, the results of each test have only been correlated with reproductive health based on observation of only a subset of the female population. Thus, it is impossible to truly assess an ovarian reserve of a particular woman with a large degree of certainty. Moreover, individual reports can provide conflicting information related to a particular woman's ovarian reserve. Accordingly, many women undergo several, if not all, of the aforementioned tests. However, because each test produces a distinct clinical report related to ovarian reserve, it is very difficult to put the individual test results into an overall context of ovarian reserve.

Therefore, the test results do not provide clinicians with clear guidance when providing women with individual reproductive health prognoses and corresponding fertility treatments. Consequently, the uncertainty associated with current tools for analyzing and reporting ovarian reserve tests provides additional emotional stress to couples struggling to achieve pregnancy. This stress can then exacerbate infertility problems as stress is considered to play a role in up to 30% of all infertility problems.

### SUMMARY

Embodiments of the present disclosure relate to an ovarian reserve measurement tool that provides patients and clinicians with a comprehensible ovarian reserve score. Notably, the ovarian reserve score provides a composite assessment of a patient's ovarian reserve based on results from a plurality of disparate ovarian reserve tests. Accordingly, the ovarian reserve score provides a reproductive health measurement that places the individual test results into an overall context of ovarian reserve. As a result, the ovarian reserve score is free from ambiguities associated with individually interpreting multiple test results.

Advantageously, the ovarian reserve measurement tool enables a clinician to confidently determine a patient's reproductive health, corresponding treatment plan, and prognosis because the ovarian reserve score does not include the uncertainties associated with current approaches of determining ovarian reserve. Consequently, embodiments of the present disclosure advantageously remove any patient stress associated with such uncertainty in a treatment plan and prognosis. As a result, such stress can no longer exacerbate any of the patient's infertility problems.

In particular, the ovarian measurement tool correlates ovarian reserve measurements from a plurality of disparate of ovarian reserve tests. The measurement tool uses the correlation to identify a relative importance of each measurement in determining a patient's ovarian reserve. In one aspect, the measurement tool utilizes a modeling technique to correlate the ovarian reserve measurements. In some examples, the modeling technique is based on the plurality of disparate ovarian reserve tests from which the ovarian reserve measurements are generated. Additionally, measurement tool uses the modeling technique to select and produce a data structure that enables processing of ovarian reserve measurements according to the correlation. The data structure processes the reproductive health data to provide a patient specific ovarian reserve score. Advantageously, a format of the date structure is selected to optimize computing resources required to process the ovarian reserve measurements.

In one embodiment, an ovarian characteristic measurement system comprises a data interface and an ovarian characteristic data structure stored in memory. The data interface is configured to receive a set of reproductive health data of a patient. The ovarian characteristic data structure is configured to process the set of reproductive health data based on an ovarian characteristic data model. The ovarian characteristic data model defines instructions for processing the reproductive health data. For instance, the data model provides a correlation between the types of the reproductive health data. The data model uses the correlation to identify a relative weight metric (e.g., importance) of each type of the reproductive health data with respect to an overall value of the ovarian characteristic. As such, the ovarian characteristic data structure is further configured to determine a value of the ovarian characteristic of the patient based on the processing of the set of the reproductive health data. A format of the ovarian data structure is selected to optimize computing resources required to process the set of reproductive health data.

In some aspects, the data interface can include a data filter for formatting the set of reproductive health data for input into the ovarian reserve data structure. Additionally, he set of reproductive health data can comprise a result from at least one test administered to measure the patient's ovarian characteristic.

The test(s) can include at least one of or any combination of: an Anti-Müllerian hormone (AMH) test, a basal antral follicle count (BAFC) test, a follicle stimulating hormone (FSH) test, a luteinizing hormone (LH) test, an estradiol estrogen (E2) test.

The ovarian characteristic being measures can be an ovarian reserve of a patient. The ovarian characteristic model can be generated from an identification of a correlation between possible values of the result and the ovarian reserve. Further, the ovarian reserve value can fall within a scale defining a fertility potential, the scale having an upper limit, a lower limit, and intermediary values.

In other aspects, the test(s) can include at least one of or any combination of an estradiol estrogen (E2) test at surge, a number of patient eggs retrieved, a number of useable embryos, a number of metaphase II (MII) embryos, and 2 pronuclear (2PN) stage embryos. Accordingly, the ovarian characteristic can be an ovarian response.

Another embodiment of the present disclosure is a method for measuring an ovarian characteristic of a patient. The method comprises receiving a set of reproductive health data of a patient. In addition, the method comprises processing the set of reproductive health data according to an ovarian characteristic data structure stored in memory. The data structure is generated from an ovarian characteristic data model that defines a relationship between the set of reproductive health data in order to yield a measurable value of the ovarian characteristic. For instance, the data model provides a correlation between the types of the reproductive health data. The data model uses the correlation to identify a relative weight metric (e.g., importance) of each type of the reproductive health data with respect to an overall value of the ovarian characteristic. Accordingly, the method further comprises determining a value of the ovarian characteristic of the patient based on the processing of the set of reproductive health data. In order to process efficiently process the reproductive health data, a format of the data structure is selected to optimize computing resources required to process the set of reproductive health data.

In some aspects, the method includes receiving the data at a data interface comprising a data filter for formatting the set of reproductive health data for input into the ovarian reserve data structure.

In some aspects, the method includes building the ovarian characteristic model by determining a correlation between possible values of the result and the ovarian reserve.

### BRIEF DESCRIPTION OF THE DRAWINGS

The foregoing will be apparent from the following more particular description of example embodiments of the present disclosure, as illustrated in the accompanying drawings in which like reference characters refer to the same parts throughout the different views. The drawings are not necessarily to scale, emphasis instead being placed upon illustrating embodiments of the present disclosure.

FIG. 1 illustrates an example environment in which an ovarian characteristic measurement system operates in accordance with an example embodiment of the present disclosure.

FIG. 2 illustrates relationships between genotypes of differing levels of FMR1 expression and ovarian health.

FIG. 3 is a flow diagram of a method for determining a value of an ovarian characteristic in accordance with an example embodiment of the present disclosure.

FIG. 4 is a flow diagram of a method for generating and ovarian characteristic model in accordance with an example embodiment of the present disclosure.

FIG. 5 illustrates an example data graph of an ovarian reserve model in accordance with an example embodiment of the present disclosure.

FIG. 6 is a block diagram of an ovarian reserve system in accordance with an example embodiment of the present disclosure.

### DETAILED DESCRIPTION

As a woman ages, her supply of eggs gradually declines over time until the eggs are depleted at menopause. Although it is expected for the ovary to age in a certain way, there are times when it doesn't behave as predicted. Accordingly, screening for ovarian reserve is a fundamental part of an initial evaluation for infertility patients of any age.

The term “ovarian reserve” refers to a woman's current supply of eggs, and is closely associated with reproductive potential. In general, the greater the number of remaining eggs, the better the chance for conception. Conversely, low ovarian reserve greatly diminishes a patient's chances for conception.

Since a woman's chronological age is the single most important factor in predicting a couple's reproductive potential, age has often guided infertility treatment choices. However, age alone doesn't tell the whole story. Consequently, researchers have developed (and are continuing to develop) more refined methods of predicting a woman's response to infertility treatment. Some of the more sophisticated tools include determining the woman's ovarian reserve by making and analyzing measurements of Anti-Müllerian hormone (AMH), basal antral follicle count (BAFC), and day three levels of follicle-stimulating hormone (FSH), luteinizing hormone (LH), and estrogen (i.e., estradiol (E2)). Additionally, certain genetic data can also be used to assess fertility potential and/or ovarian reserve. This can include, but is not limited to, gene expression signatures of tissues constituting the reproductive tract, levels of one or more particular proteins circulating in the blood or making up other body fluids including uterine secretions, and genetic variants.

Even though several sophisticated tools exist for measuring ovarian reserve, most fall short of what we consider ideal sensitivity and specificity. Also, how best to interpret ovarian reserve tests is controversial, since clinical experience with these tests is still evolving.

Embodiments of the present disclosure relate to an ovarian reserve measurement tool that provides patients and clinicians with a comprehensible ovarian reserve score. Notably, the ovarian reserve score provides a composite assessment of a patient's ovarian reserve based on results from a plurality of disparate ovarian reserve tests. Accordingly, the ovarian reserve score provides a reproductive health measurement that places the individual test results into an overall context of ovarian reserve. As a result, the ovarian reserve score is free from ambiguities associated with individually interpreting multiple test results.

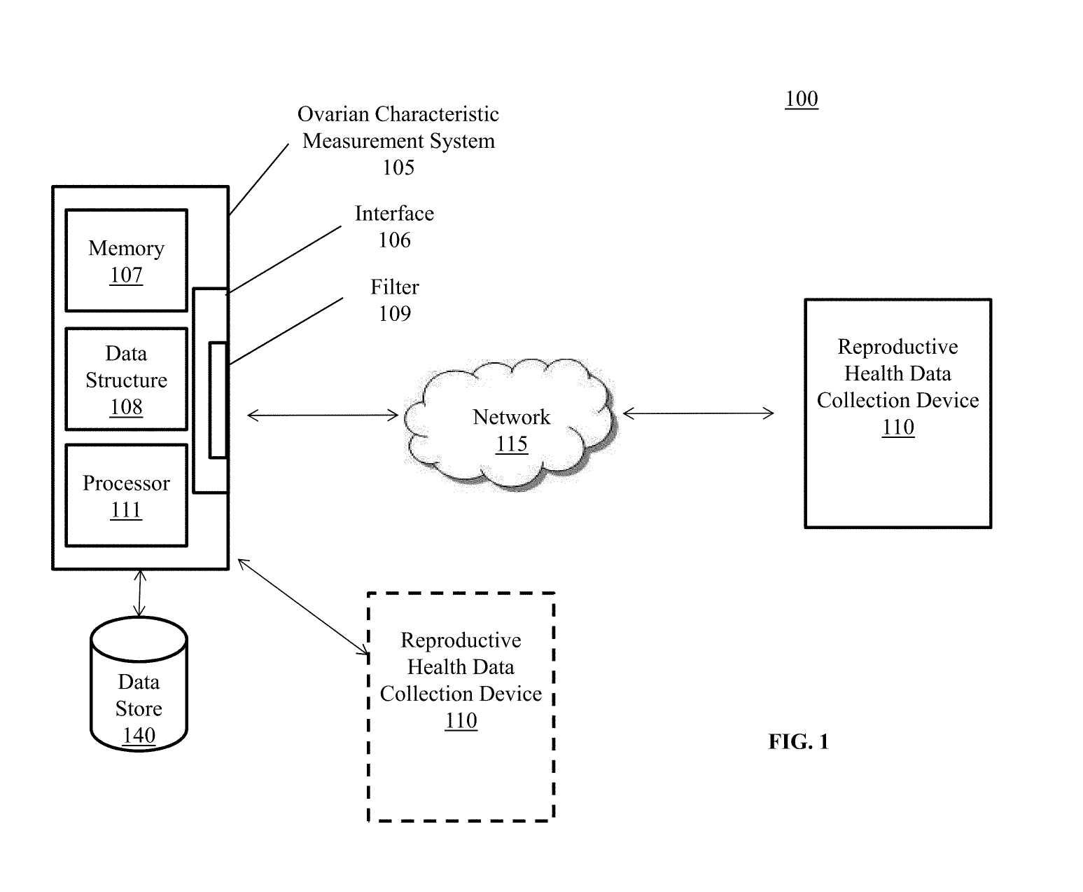

FIG. 1 illustrates an example environment 100 in which an ovarian characteristic (e.g., ovarian reserve) measurement system 105 operates in accordance with an example embodiment of the present disclosure. The ovarian reserve measurement system 105 is in communication with a reproductive health data collection device 110. In particular, the ovarian reserve measurement system 105 includes an interface 106 configured to receive and process reproductive health data collected by the collection device 110. For example, the collection device 110 can be any type of medical equipment used to obtain measurements for determining ovarian reserve (e.g., measurements of Anti-Müllerian hormone (AMH), basal antral follicle count (BAFC), and day three levels of follicle-stimulating hormone (FSH), luteinizing hormone (LH), estrogen (i.e., estradiol (E2))), and genetic data.

The ovarian reserve measurement system 105 and the reproductive health data collection device 110 can be communicatively coupled via a network 115. The network 115 can be a local area network (LAN), wide area network (WAN) (e.g., the Internet), or an ad-hoc network. In some examples, the health data collection device 110 and the ovarian reserve measurement system 105 can be communicatively coupled via a wired communications link or a near-field wireless link (e.g., Wi-Fi and/or Bluetooth).

In response to receiving the reproductive health data, the ovarian reserve measurement system 105 can filter the data using a filter 109. The filter 109 is configured to format the reproductive health data for processing by the data structure 108 (e.g., an ovarian reserve data structure) which can be stored in data store 140 and executed by processor 111. For example, the filter 109 ensures the structure of all data being input into the data structure corresponds to a format of the data structure. Memory 107 can be random access memory (RAM) used by the processor 111 to store instructions for processing the data structure 108 in order to increase the general speed of the ovarian reserve measurement system 101.

The data structure 108 is configured to process the ovarian reserve measurements and provide a composite ovarian reserve score. In particular, the ovarian reserve data structure 108 is configured to process the set of ovarian reserve measurements based on an ovarian reserve data model. The ovarian reserve data model provides a correlation between the types of ovarian reserve measurements. The data model uses the correlation to identify a relative weight (e.g., importance) of each of the types of ovarian reserve measurements with respect to an overall ovarian reserve measurement. As such, the processor 111 processes the received measurements through a processing pipeline defined by the ovarian reserve data structure to determine a composite value of ovarian reserve. Advantageously, the ovarian reserve measurement system 105 provides a single ovarian reserve score that enables a clinician to confidently determine a patient's reproductive health, corresponding treatment plan, and prognosis because the ovarian reserve score does not include the uncertainties associated with current approaches of determining ovarian reserve.

The ovarian reserve measurement system 105 utilizes a modeling technique such as structural equation modeling (SEM) to generate the ovarian reserve data model and corresponding data structure 108. Methods of structural equation modeling are described, for example, in Bollen (Structural Equations with Latent Variables, John Wiley & Sons, Inc., 1989, New York); and Kline (Principles and Practice of Structural Equation Modeling, 4th Ed., Guilford, 2015, New York), the content of each of with is hereby incorporated by reference in its entirety. Notably, SEM includes a diverse set of mathematical models, computer algorithms, and statistical methods that fit networks of constructs to data. For instance, SEM includes confirmatory factor analysis, path analysis, partial least squares path modeling, LISREL and latent growth modeling. SEM is commonly used to assess unobservable ‘latent’ constructs. Particularly, SEM invokes a measurement model that defines latent variables (e.g., ovarian reserve) using one or more observed variables (e.g., measurements of Anti-Müllerian hormone (AMH), basal antral follicle count (BAFC), and day three levels of follicle-stimulating hormone (FSH), luteinizing hormone (LH), estrogen (i.e., estradiol (E2))), and genetic data.

Genetics can represent one additional contributory mechanism for the decline in ovarian reserve. Age-related decline in ovarian reserve appears to be associated with decreased expression of genes involved in DNA repair by homologous recombination such as BRCA1, MRE11, Rad51 and ATM. However, the expression levels of these genes can be low (or otherwise aberrant) in women of normal reproductive age, perhaps due to an inherent pathology or the result of an environmental factor. This in turn may lead to such women having a lower than normal ovarian reserve. The expression levels of these genes can therefore be useful in predicting ovarian reserve or predicting future low ovarian reserve. The use of genetic data in this way need not be limited to expression data. In the same way that levels of AMH are a biochemical reflection of the size of the pool of oocytes, it is possible that the serum levels of other markers, or levels of those markers in the follicular fluid, can be used because they are reflective of ovarian reserve, or they affect ovarian reserve, and in both of these respects are predictive of it. Examples of proteins that could be measured include lipoproteins like cholesterol and other steroidogenic substrates: steroidogenic enzymes such as 21-hydroxylase, aromatase, 17 alpha-hydroxylase; cytokines like TNFa, interleukins 1A, 6, 11, 18,10.

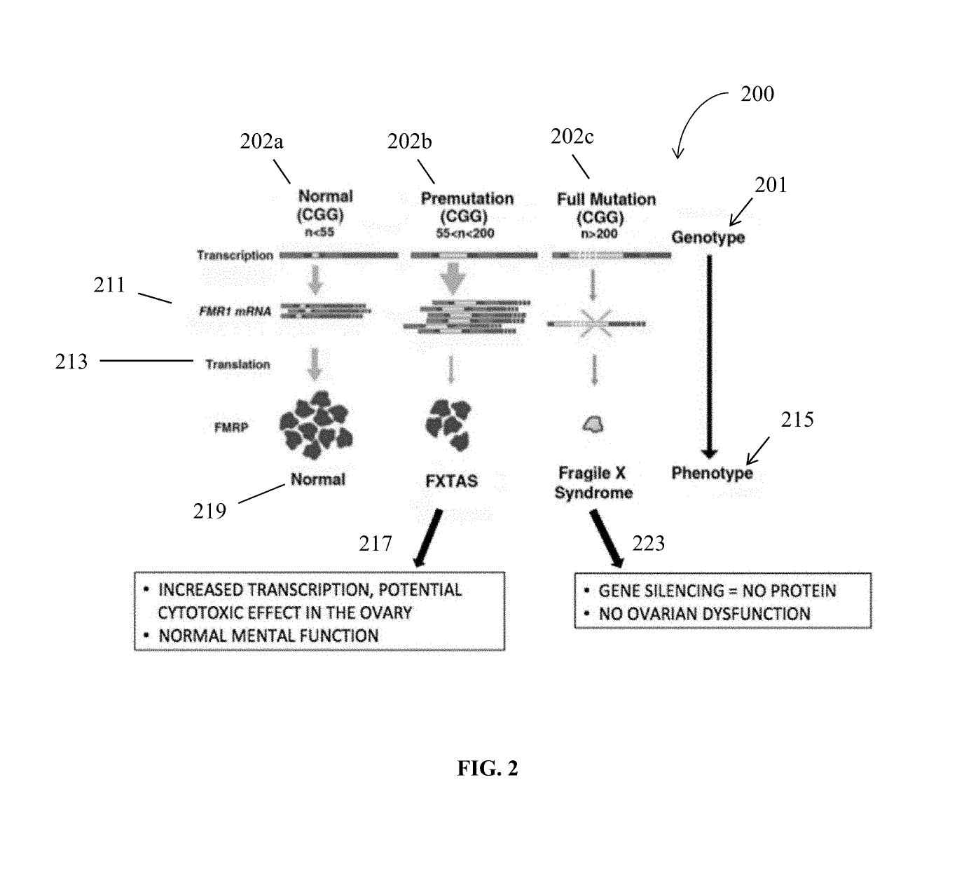

Genetic data need not be limited to expression or levels of protein-genetic variants can also be examined, and could potentially increase the strength of associations of particular factors, if for example genotypic information is added as a field within a factor (e.g. those identified in Table 2a below). FIG. 2 illustrates an example of a gene that could be examined for variation and illustrates an example relationship model 200 between genotypes 201 of fragile X mental retardation 1 (FMR1) and phenotypes 215 related to ovarian health. FMR1 encodes a protein, fragile X mental retardation protein (FMRP), most commonly found in the brain. This protein is essential for normal cognitive development and female reproductive function. The gene contains a highly polymorphic CGG repeat in exon 1. The number of repeats 202a-c varies between individuals, and different numbers of these repeats are associated with different phenotypes such as fragile X syndrome 223, intellectual disability, premature ovarian failure 217, autism, Parkinson's disease, developmental delays and other cognitive deficits. Table 1 below illustrates example relationships between varying levels CGG repeats 202a-c and their respective relationships with both cognitive and fertility phenotypes 215. FIG. 2 illustrates a relationship model 200 demonstrating that if a number of CGG repeats 202a-c is within a particular threshold, the number of FMR1 mRNA 211 transcripts is predicted to have a cytotoxic effect in the ovary and thus the variant also has an effect on ovarian function and ovarian reserve. Thus, since the number of CGG repeats 202a-c in FMR1 has a mechanistic link to ovarian reserve, it can be used to predict ovarian reserve.

TABLE 1
Relationships between CGG repeat and phenotype.

| # CGG | Classification | | Ovarian reserve/Fertility related |
|---|---|---|---|
| Repeats | of mutation | Cognitive phenotype | phenotype |
| >200 | Full mutation | Fragile X syndrome (FXS) in the | Fragile X-associated primary |
| | (202c) | majority of boys and in some girls | ovarian insufficiency (FXPOI) |
| | | carrying the mutation | |
| 55-199 | Premutation | Late-onset tremor/ataxia syndrome, | Increased risk of primary ovarian |
| | (202b) | termed fragile X-associated tremor | insufficiency (POI); A very specific |
| | | ataxia syndrome (FXTAS); Also | sub-genotype of FMR1 has been |
| | | increased risk of expansion to full | found to be associated |
| | | mutation during transmission to | with polycystic ovarian |
| | | offspring | syndrome (PCOS) |
| 45-54 | Intermediate | Minor genetic instability | Greater prevalence of subfertility; |
| | | | Menstrual cycle anomalies; Earlier |
| | | | than anticipated menopause (by ~7 |
| | | | years); and Primary ovarian |
| | | | insufficiency. |
| <45 | Normal (202a) | No association with abnormal | No documented association with |
| | | phenotype | phenotype, but could still be |
| | | | predictive of ovarian reserve. (219) |

A skilled artisan understands that the relationship principle demonstrated in FIG. 2 can be applied to any gene associated with ovarian reserve mechanisms. Thus mutations in those genes could be added as fields within factors (e.g. those in Tables 2a-b) to calculate composite ovarian reserve scores more precisely.

Referring back to FIG. 1, the ovarian reserve measurement system 105 can use SEM to generate an ovarian reserve data model that determines a correlation between the aforementioned distinct indirect ovarian reserve measurements. In particular, the model identifies a relative importance and/or weight of each measurement with respect to an ovarian reserve score. Further, the model defines a scale of values of ovarian reserve. The scale has an upper limit, a lower limit, and intermediate values. In some embodiments, an ovarian reserve score having a value at or near the upper limit can indicate a good ovarian reserve level (e.g., one indicating a healthy level of fertility), while a score at or near to the lower limit can indicate a bad ovarian reserve level (e.g., one indicating an unhealthy level of fertility or infertility). In other embodiments, an ovarian reserve score having a value at or near the upper limit can indicate a bad ovarian reserve level, while a score at or near to the lower limit can indicate a good ovarian reserve level.

One skilled in the art understands that other known or yet to be known modeling techniques can be used. For example, principal component analysis can be used to project a high dimensional data set (e.g., the above referenced ovarian reserve measurements) into a lower-dimensional space such that dimensions that were highly correlated in the original space are reduced to a lower number of dimensions in the transformed space; in this fashion the transformed dimensions in PCA may be used to represent latent variables (e.g., an ovarian reserve) that are correlated with the original (untransformed) observed variables. Methods of PCA are described in Jolliffe (Principal Component Analysis, 2^{nd} Ed., Springer, 2002, New York), the content of which is hereby incorporated by reference in its entirety. There are many related techniques of component analysis and matrix factorization that could be substituted for PCA, such as incremental PCA, kernel PCA, factor analysis, and non-negative matrix factorization; all of these may be used with the methods of the invention.

Another modeling technique can include using autoencoders. Autoencoders are artificial neural networks that can be used to perform nonlinear dimensional reduction. As with PCA, the reduced set of dimensions produced by the autoencoder may be used to represent latent variables that are correlated with the original observed variables. Methods of constructing and using autoencoders are described in Goodfellow and Bengio (Deep Learning, MIT Press, 2016, Cambridge, Mass., Chapter 14), which is hereby incorporated by reference.

Other techniques or algorithms are known to those of skill in the art, and may be used with methods of the invention, such as factor analysis, partial least squares regression, manifold learning algorithms (isomap, locally linear embedding, and others), Bayes nets, and probabilistic graphical models.

Based on the ovarian reserve data model, the processor 111 generates the ovarian reserve data structure 108. The processor 111 uses the data structure 108 to process incoming ovarian reserve measurements to provide an ovarian reserve score as defined by the ovarian reserve model. Advantageously, the processor generates a data structure that optimizes computing resources. For example, the data structure can be a graph data type. A graph data structure includes a set of vertices or nodes or points, together with a set of unordered pairs of these vertices for an undirected graph or a set of ordered pairs for a directed graph. The pairs are known as edges, arc, or lines for an undirected graph and as arrows, directed edges, directed arcs, or directed lines for a directed graph. The graph data structure may also associate some edge value, such as a numeric attribute, to each edge.

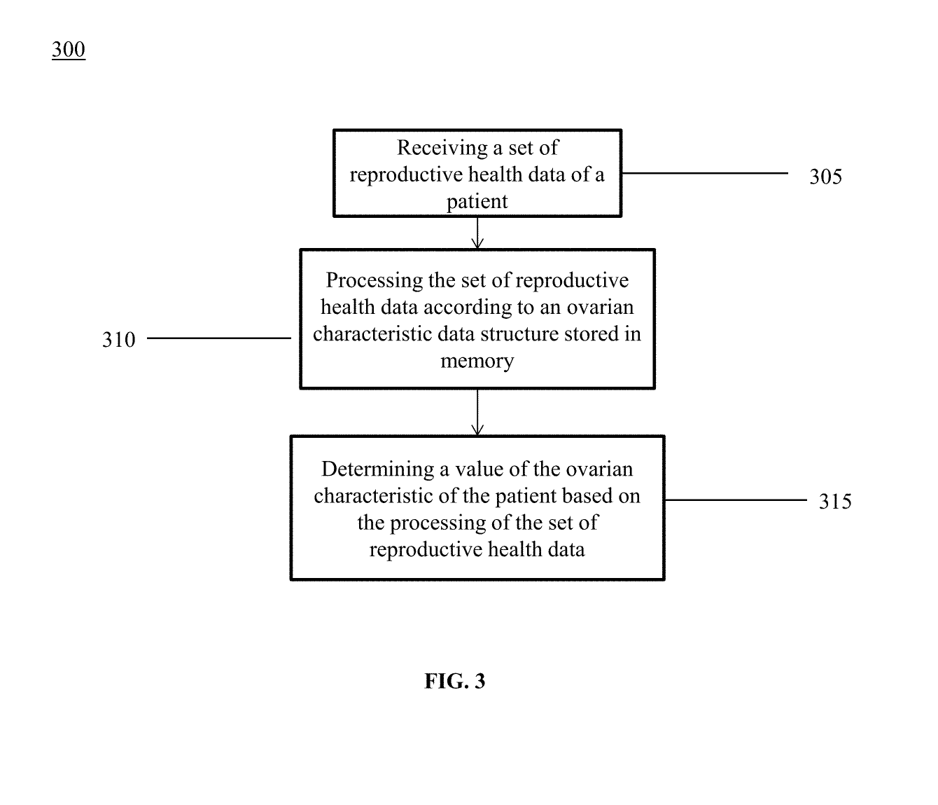

FIG. 3 is a flow diagram of a method 300 for determining a value of an ovarian characteristic in accordance with an example embodiment of the present disclosure. The method 300, at 305, includes receiving a set of reproductive head data of a patient. The reproductive health data can be the indirect ovarian reserve measurements discussed herein. At 310, the method 300 includes processing the data according to an ovarian characteristic data structure (e.g., the ovarian reserve data structure 108 of FIG. 1) stored in memory. The method 300, at 315, includes determining a value of the ovarian reserve (e.g., an ovarian reserve score) of the patient.

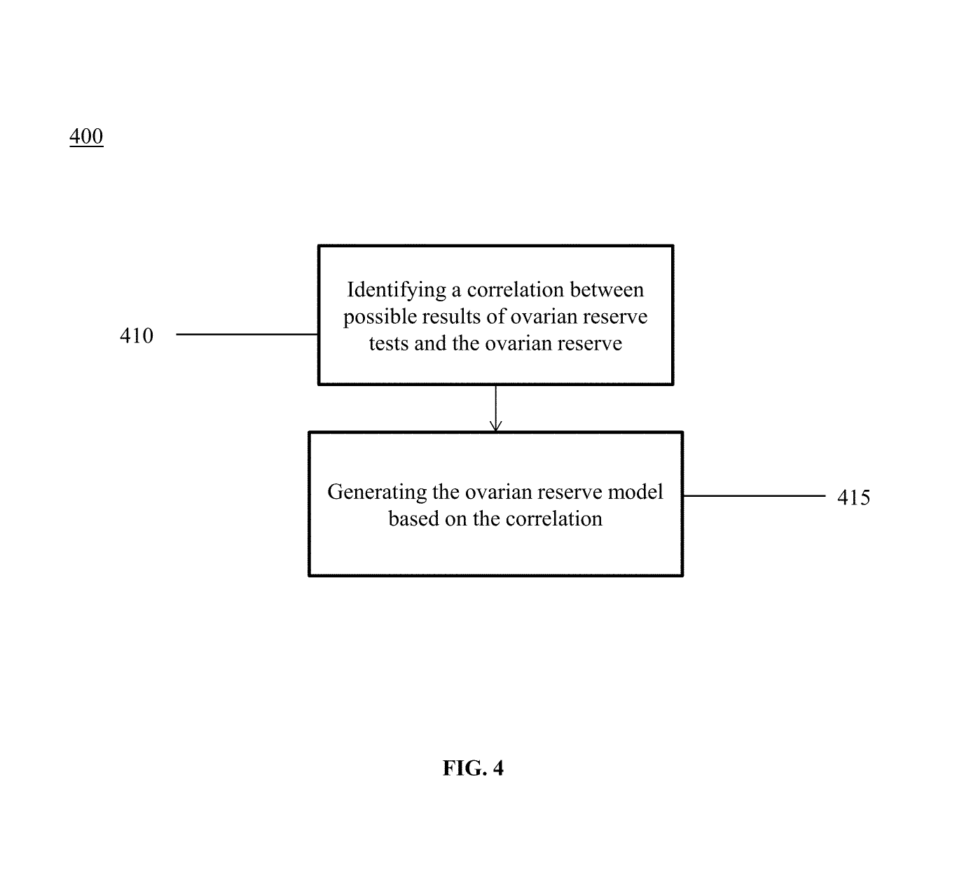

FIG. 4 is a flow diagram of a method 400 for generating and ovarian characteristic model (e.g., an ovarian reserve model) in accordance with an example embodiment of the present disclosure. The method 400, at 410, comprises identifying a correlation between possible results of indirect ovarian reserve tests and ovarian reserve. At 415, the method 300 includes generating the ovarian reserve model based on the identified correlation.

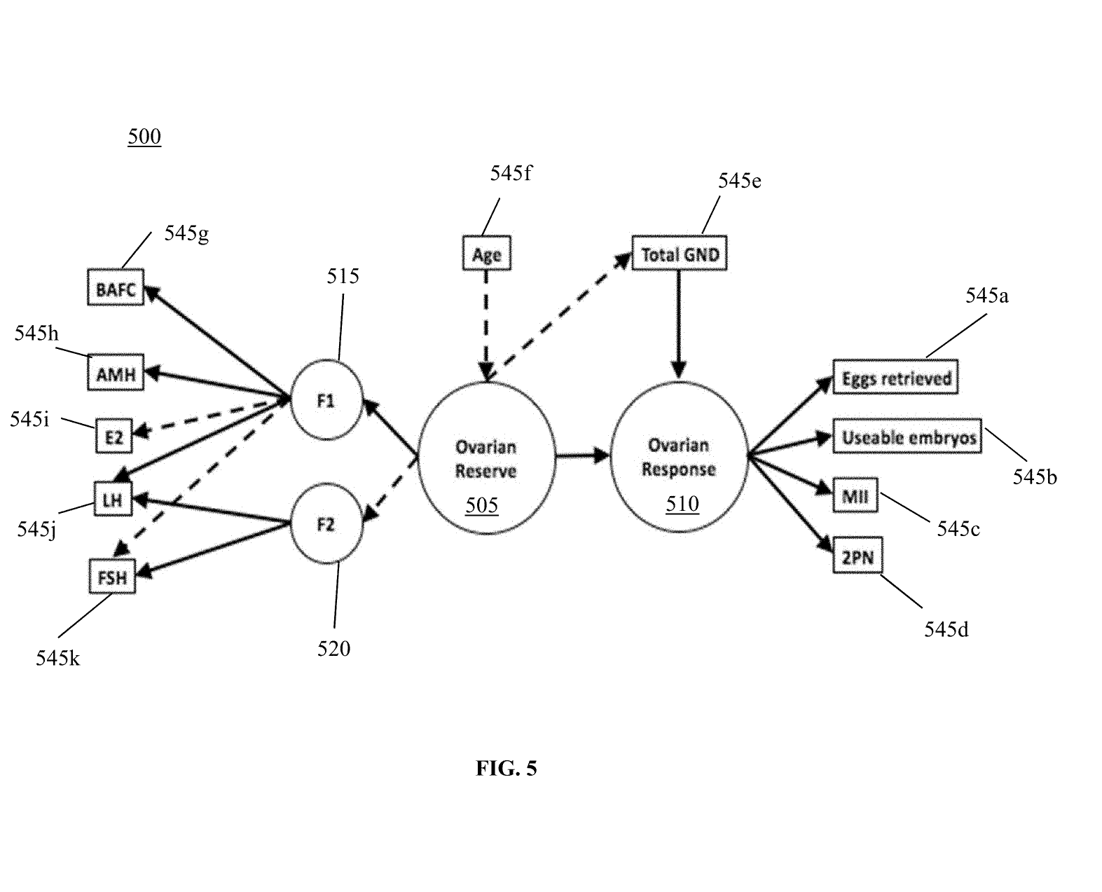

FIG. 5 illustrates an example structural equation model path diagram 500 of an ovarian reserve model in accordance with an example embodiment of the present disclosure. The path diagram 500 includes a set of observed variables 545a-k. In addition, the path includes a set of unobserved variables (latent) factors 505, 510, 515, 520. Arrows point out of latent factors 505, 510, 515, and 520 towards the observed 545a-k variables which measure these latent factors. Arrows pointing out of the observed variables indicate hypothesized causal relationships. Dotted arrows indicate a negative correlation and solid arrows indicate a positive correlation.

For example, the equation path diagram 500 represents an example experiment used to develop an ovarian reserve scale. The example experiment is as follows.

### Statistical Analysis:

71,011 in-vitro fertilization (IVF) treatment cycles were analyzed to develop an ovarian reserve scale measured by Anti-Müllerian hormone (AMH) 545h, basal antral follicle count (BAFC) 545g, and day three levels of follicle-stimulating hormone (FSH) 545k, luteinizing hormone (LH) 545j, and estrogen (E2) 545i. Exploratory factor analysis (EFA) was used to explore a covariance structure of the measures and confirmatory factor analysis (CFA) was used to formally test the hypothesis that each of these metrics measure the same common latent factor (i.e., ovarian reserve 505).

Structural equation modelling (SEM) was used to evaluate the relationship between age 545f, total gonadotropins administered 545e, ovarian reserve 505, and ovarian response 510 to IVF. Ovarian response 510 was defined using a latent factor which was measured by the number of eggs retrieved 545a, the number of useable embryos 545b, and the number of MII 545c and 2PN embryos 545d.

For EFA, a parallel analysis of the eigenvalues was used to determine the number of latent factors to retain. For CFA and SEM, model fit was evaluated using several fit indices including CFI, TLI, RMSEA, and SRMR. Good fitting models typically have indices such chat CFI>0.95, TLI>0.95, RMSEA<0.08, and SRMR<0.07.

### Results:

A parallel analysis of the eigenvalues from the EFA showed that a two factor model best described the covariance structure between AMH 545h, BAFC 545g, and day three levels of LH 545j, FSH 545k, and E2 545i. The best fitting SEM corresponded to a two factor measurement model with AMH 545h, BAFC 545g, and day three levels of LH 545j, FSH 545k, and E2 545i loading on the first factor 515, and day 3 LH 545j and FSH 545k loading on the second factor 520. A second-order latent factor was introduced, which was measured by factor F1 515 and factor F2 520 and thereby combined their values into a univariate measure of ovarian reserve 505. Lastly, ovarian response 510 was a latent factor measured by the number of eggs retrieved 545a, the number of useable embryos 545b, and the number of MII 445c and 2PN embryos 545d. The model fit indices were all within the good range (CFI=0.96, TLI=0.95, RMSEA=0.06, SRMR=0.04).

AMH 545h, BAFC 545g, and LH 545j positively loaded on Factor F1 515; E2 545i and FSH 545k negatively loaded on Factor F1 515. In particular, AMH 545h and BAFC 545g loaded the most strongly on this factor 515 and had very similar loadings, indicating that they contribute a similar amount of information to the measurement of Factor F1 515. LH 545j had the third strongest loading on factor F1 515 followed by FSH 545k and lastly E2 545i. Both LH 545j and FSH 545k positively loaded on Factor F2 520. Factors F1 515 and F2 520 positively and negatively loaded on the second-order factor. The number of eggs retrieved 545a, the number of useable embryos 545b, and the number of MII and 2PN embryos 545c-d all significantly loaded onto the latent factor defining ovarian response 510. Standardized estimates of all factor loadings of this model are shown in Table 2a.

The structural component of the model revealed that the first factor decreased as a function of age and the second increased as a function of age. Lastly, the number of eggs retrieved was positively correlated with the second-order latent factor (ovarian reserve 505) and negatively correlated with age, even after controlling for ovarian reserve. Parameter estimates form the structural components are shown in Table 2b.

TABLE 2a
Standardized factor loadings of structural component of SEM.

| | Standardized Factor | |
|---|---|---|
| | Loading (lambda) | P-value |
| Factor 1 measured by: | | |
| AMH | 0.772 | <0.001 |
| BAFC | 0.817 | <0.001 |
| E2 | −0.069 | <0.001 |
| LH | 0.274 | <0.001 |
| FSH | −0.179 | <0.001 |
| Factor 2 measured by: | | |
| LH | 0.388 | <0.001 |
| FSH | 0.833 | <0.001 |
| Ovarian reserve | | |
| measured by: | | |
| Factor 1 | 0.762 | <0.001 |
| Factor 2 | −0.225 | <0.001 |
| Ovarian response | | |
| measured by: | | |
| M2 | 0.777 | <0.001 |
| PN2 | 0.936 | <0.001 |
| Useable embryos | 0.681 | <0.001 |
| Eggs retrieved | 0.903 | <0.001 |

In Table 2a, the loadings closest in magnitude to 1 have stronger associations with the latent factor. All of the analyzed measures of ovarian reserve loaded significantly on actor 1, whereas LH and FSH only loaded on Factor 2. The second-order Factor 3 (ovarian reserve) positively correlated with Factor 1 and negatively correlated with Factor 2.

TABLE 2b
Structural regression standardized coefficients.

| | Standardized Coefficient | P-value |
|---|---|---|
| Ovarian reserve | | |
| regressed on: | | |
| Patient age | −0.546 | <0.001 |
| Total GND | | |
| regressed on: | | |
| Ovarian reserve | −0.559 | <0.001 |
| Ovarian Response | | |
| regressed on: | | |
| Ovarian reserve | 0.803 | <0.001 |
| Total GND | 0.405 | <0.001 |

Aspects of the invention described herein can be performed using any type of computing device, such as a computer, that includes a processor, e.g., a central processing unit, or any combination of computing devices where each device performs at least part of the process or method. In some embodiments, systems and methods described herein may be performed with a handheld device, e.g., a smart tablet, a smart phone, or a specialty device produced for the system.

Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

Processors suitable for the execution of computer program include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through network by any form or medium of digital data communication, e.g., a communication network. For example, the reference set of data may be stored at a remote location and the computer communicates across a network to access the reference set to compare data derived from the female subject to the reference set. In other embodiments, however, the reference set is stored locally within the computer and the computer accesses the reference set within the CPU to compare subject data to the reference set. Examples of communication networks include cell network (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Java, ActiveX, HTML5, Visual Basic, or JavaScript.

A computer program does not necessarily correspond to a file. A program can be stored in a file or a portion of file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over a network (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

Suitable computing devices typically include mass memory, at least one graphical user interface, at least one display device, and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, radiofrequency identification tags or chips, or any other medium which can be used to store the desired information and which can be accessed by a computing device.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

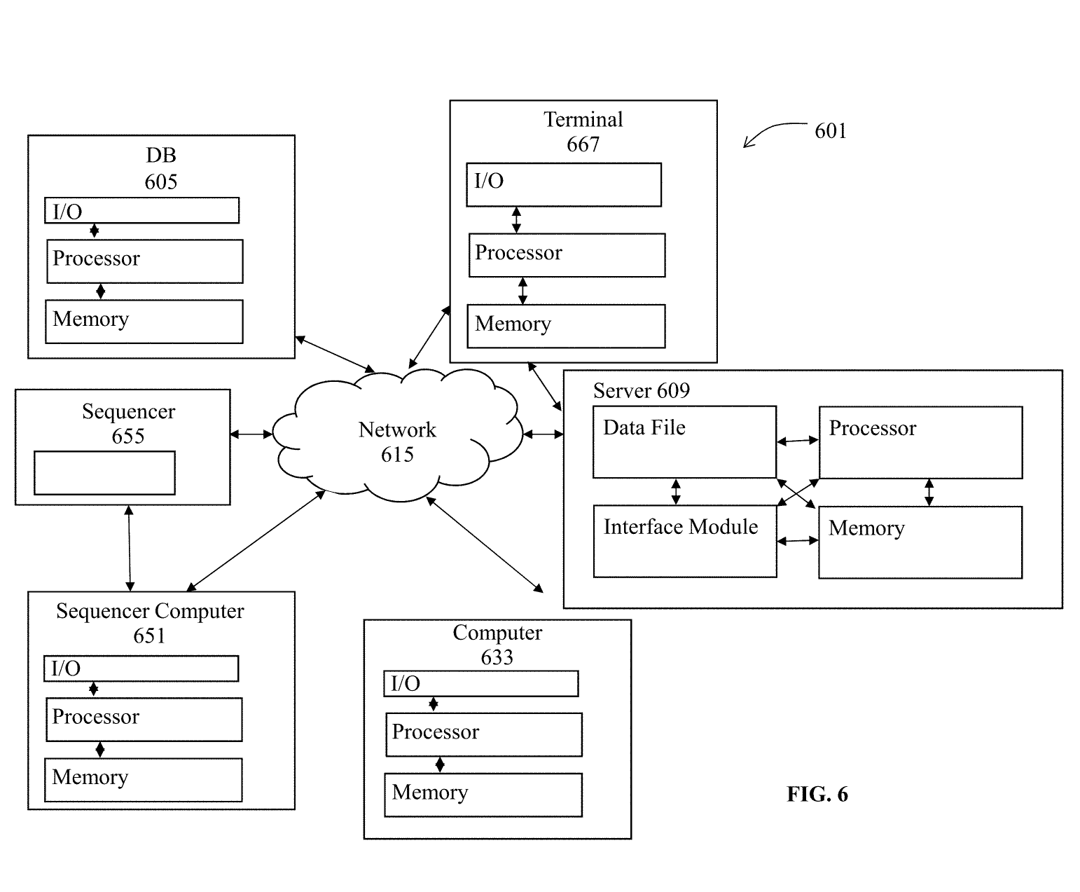

In an exemplary embodiment shown in FIG. 6, system 601 can include a computer 633 (e.g., laptop, desktop, or tablet). The computer 633 may be configured to communicate across a network 615. Computer 633 includes one or more processor and memory as well as an input/output mechanism. Where methods of the invention employ a client/server architecture, any steps of methods of the invention may be performed using server 609, which includes one or more of processor and memory, capable of obtaining data, instructions, etc., or providing results via interface module or providing results as a file. Server 609 may be engaged over network 615 through computer 633 or terminal 667, or server 615 may be directly connected to terminal 667, including one or more processor and memory, as well as input/output mechanism. In some embodiments, systems include an instrument 655 for obtaining sequencing data, which may be coupled to a sequencer computer 651 for initial processing of sequence reads

Memory according to the invention can include a machine-readable medium on which is stored one or more sets of instructions (e.g., software) embodying any one or more of the methodologies or functions described herein. The software may also reside, completely or at least partially, within the main memory and/or within the processor during execution thereof by the computer system, the main memory and the processor also constituting machine-readable media. The software may further be transmitted or received over a network via the network interface device.

Other embodiments are within the scope and spirit of the invention. For example, due to the nature of software, functions described above can be implemented using software, hardware, firmware, hardwiring, or combinations of any of these. Features implementing functions can also be physically located at various positions, including being distributed such that portions of functions are implemented at different physical locations.

While the present disclosure has been particularly shown and described with references to example embodiments thereof, it will be understood by those skilled in the art that various changes in form and details may be made therein without departing from the scope of the present disclosure encompassed by the appended claims. Accordingly, all changes which come within the meaning and range of equivalency of the claims (e.g., any element(s) with the claims or any of the claims as a whole) are intended to be embraced therein.

What is claimed is:

1. An ovarian characteristic measurement system, the system comprising:
a data interface configured to receive a set of reproductive health data of a patient; and
an ovarian characteristic data structure stored in memory and configured to:
process the set of reproductive health data based on an ovarian characteristic data model, the ovarian characteristic data model defining a processing of the reproductive health data, wherein a format of the ovarian data structure is selected to optimize computing resources required to process the set of reproductive health data, and
determine a value of the ovarian characteristic of the patient based on the processing of the set of reproductive health data.

2. The system of claim 1 wherein the data interface includes a data filter for formatting the set of reproductive health data for input into the ovarian reserve data structure.

3. The system of claim 1 wherein the set of reproductive health data comprises a result from at least one test administered to measure the patient's ovarian characteristic.

4. The system of claim 3 wherein the at least one test includes at least one of or any combination of: an Anti-Müllerian hormone (AMH) test, a basal antral follicle count (BAFC) test, a follicle stimulating hormone (FSH) test, a luteinizing hormone (LH) test, an estradiol estrogen (E2) test, and genetic data.

5. The system of claim 4 wherein the ovarian characteristic is an ovarian reserve.

6. The system of claim 5 wherein the ovarian characteristic model is generated from an identification of a correlation between possible values of the result and the ovarian reserve.

7. The system of claim 6 wherein the ovarian reserve value falls within a scale defining a fertility potential, the scale having an upper limit, a lower limit, and intermediary values.

8. The system of claim 3 wherein the at least one test includes at least one of or any combination of an estradiol estrogen (E2) test at surge, a number of patient eggs retrieved, a number of useable embryos, a number of metaphase II (MII) embryos, and 2 pronuclear (2PN) stage embryos.

9. The system of claim 8 wherein the ovarian characteristic is an ovarian response.

10. The system of claim 9 wherein the ovarian characteristic model is generated from an identification of a correlation between possible values of the result and the ovarian response.

11. The system of claim 10 wherein the value of the ovarian response falls within a scale defining a response to ovarian stimulation, the scale having an upper limit, a lower limit, and intermediary values.

12. A method for measuring an ovarian characteristic of a patient, the method comprising:
receiving a set of reproductive health data of a patient;
processing the set of reproductive health data according to an ovarian characteristic data structure stored in memory, wherein the data structure is generated from an ovarian characteristic data model defining the ovarian characteristic and wherein a format of the data structure is selected to optimize computing resources required to process the set of reproductive health data; and
determining a value of the ovarian characteristic of the patient based on the processing of the set of reproductive health data.

13. The method of claim 12 wherein receiving the set of reproductive health data includes receiving the data at a data interface comprising a data filter for formatting the set of reproductive health data for input into the ovarian reserve data structure.

14. The method of claim 12 wherein the set of reproductive health data comprises a result from at least one test administered to measure the patient's ovarian characteristic.

15. The method of claim 14 wherein the at least one test includes at least one of or any combination of: an Anti-Müllerian hormone (AMH) test, a basal antral follicle count (BAFC) test, a follicle stimulating hormone (FSH) test, a luteinizing hormone (LH) test, an estradiol estrogen (E2) test, and genetic data.

16. The method of claim 15 wherein the ovarian characteristic is an ovarian reserve.

17. The method of claim 16 further comprising building the ovarian characteristic model by determining a correlation between possible values of the result and the ovarian reserve.

18. The method of claim 17 wherein the ovarian reserve value falls within a scale defining a fertility potential, the scale having an upper limit, a lower limit, and intermediary values.

19. The method of claim 14 wherein the at least one test includes at least one of or any combination of an estradiol estrogen (E2) test at surge, a number of patient eggs retrieved, a number of useable embryos, a number of metaphase II (MII) embryos, and 2 pronuclear (2PN) stage embryos.

20. The method of claim 19 wherein the ovarian characteristic is an ovarian response.

21. The method of claim 20 further comprising generating the ovarian characteristic model based on an identification of a correlation between possible values of the result and the ovarian response.

22. The method of claim 21 wherein the value of the ovarian response falls within a scale defining a response to ovarian stimulation, the scale having an upper limit, a lower limit, and intermediary values.
